(19) United States
(12) Patent Application Publication
(10) Pub. No.: US 2010/0233107 A1
(43) Pub. Date: Sep. 16, 2010

(54) MEDICATED TONING LOTION

(76) Inventors: JENNIFER CHANG, (US); ADAYA ROCHA, (US)

Correspondence Address: KLEINBERG & LERNER, LLP, 1875 CENTURY PARK EAST, SUITE 1150, LOS ANGELES, CA, 90067, US

(21) Appl. No.: 12/652,924

(22) Filed: Jan. 6, 2010

Related U.S. Application Data
(60) Provisional application No. 61/160,130, filed on Mar. 13, 2009.

(51) Int. Cl. A61K 8/18; A61K 31/60; A61Q 17/04

(52) U.S. Cl. 424/59; 514/159

(57) ABSTRACT

A skin lotion includes, as a principal ingredient, a crosspolymer compound with as much as 50% adsorbed Salicylic Acid which can be gradually released to the skin after application. The lotion can also include, surfactants, humectants, preservatives, moisturizers, sunblocks, antibacterials and retinol precursors. A hydrophobically modified polymer contributes a smooth feel to the lotion.

### CROSS REFERENCE TO RELATED APPLICATIONS

This is a non-provisional application corresponding to our provisional application Ser. No. 61/160,130, filed Mar. 13, 2009, the priority of which is claimed.

### STATEMENT REGARDING FEDERALLY SPONSORED RESEARCH/DEVELOPMENT

None

### REFERENCE TO MICROFICHE APPENDIX

None

### BACKGROUND OF THE INVENTION

1. Field of the Invention

The present invention relates to cosmetic preparations and, more particularly, a toning lotion containing salicylic acid.

2. Description of the Related Art

Toning lotions are among the most popular cosmetic preparations used in the United States. They generally contain water as the main ingredient plus humectants, such as glycerine, propylene glycol and, sometimes, an alcohol, such as ethyl alcohol or isopropyl alcohol, which are added to provide a cooling effect.

In recent years, alcohol-free toning lotions have gained popularity as they are perceived to be less irritating and less drying to the skin than are traditional toning lotions containing alcohol. With the increased popularity of “cosmeceuticals,” a hybrid of cosmetics and pharmaceuticals, medicated toning lotions have gained a market share in this product category.

Among medicated toning lotions, those containing salicylic acid as the main active ingredient for acne treatment are becoming increasingly popular because of their ease and comfort of use and effectiveness. Salicylic acid is recognized by the U.S. Food and Drug Administration as a safe and effective topical “over the counter” (“OTC”) drug. However, one major problem with the use of salicylic acid in a toning lotion is its limited solubility in water.

According to the Merck Index, one gram of salicylic acid can dissolve in 460 ml of water, for a solubility of approximately 0.2% by weight. Frequently, though, there is a need to increase the concentration of this active ingredient in a toning lotion to increase its efficacy.

A common way of increasing salicylic acid solubility is to incorporate an organic solvent such as ethyl alcohol, isopropyl alcohol or acetone into the toning lotion formulation. However, many such solvents, including ethyl alcohol, isopropyl alcohol and acetone, can cause dermal irritation in some users and may dry the skin, causing itchiness.

Upon application on the skin of a medicated toning lotion containing such an organic solvent, the concentration of the active ingredient will increase with evaporation of the solvent. This is not a desirable effect because the local concentration of the active ingredient on the skin may become too high and cause adverse effects, such as irritation.

Additionally, in recent years, volatile organic solvents such as ethyl alcohol, isopropyl alcohol and acetone, have been targeted for contributing to environmental pollution. Many states, including California, are actively trying to reduce their use in consumer products.

Another method for incorporating higher concentrations of salicylic acid into a toning lotion formulation is the use of a suspending agent to suspend salicylic acid crystals in the lotion. Although suspending agents such as Gum Arabic and Magnesium Aluminum Silicate (Veegum) can be used for this purpose, such agents commonly cause the end product to become quite sticky, not refreshing, and somewhat unpleasant to use. In addition, the crystal size of salicylic acid can increase significantly during the storage period, causing the formulation to become unstable during its shelf life.

### SUMMARY OF THE INVENTION

The approach of the present invention toward solving this problem is to incorporate a technique of sustained release of the active ingredient with control of rheological properties to overcome the problems of limited solubility, stickiness, and suspension instability. The result is a mild, effective and stable medicated toning lotion which is pleasant to use, and is not sticky.

This was accomplished by using a unique delivery system for active ingredients, known as The Poly-Pore® Delivery System. The delivery system utilizes a powdered allyl methacrylate crosspolymer compound sold by AMCOL International Corporation under the trade mark Poly-Pore®. The polymer adsorbs 50% salicylic acid in the void space.

The delivery system containing the active ingredient, salicylic acid is dispersed in a toning lotion with suitable surfactant and rheological modifiers to form a stable product. By adjusting ingredients in the formulation, it is possible to obtain stable products containing from 0.5% to 2.0% of salicylic acid.

The result of such formulation is a stable, low-viscosity, light-textured white lotion that is pleasant to use. Sustained release of the active ingredient, salicylic acid, allows a slow but steady delivery of the active ingredient through the diffusion from the pores in the polymer, making the product mild and nonirritating. The lack of organic solvents in the formulation also makes it non-drying and nonirritating to the skin.

### DETAILED DESCRIPTION OF THE INVENTION

Some example formulations are described below. However, it is understood that a predetermined amount of salicylic acid is incorporated in the Poly Pore polymer.

### EXAMPLE 1

| Ingredients | Weight % |
|---|---|
| Water | 89.80 |
| Glycerin | 5.00 |
| Potassium Sorbate | 0.10 |
| Allantoin | 0.10 |
| Botanical Extracts | 1.50 |
| Sepiplus 400 | 1.00 |
| Poly-Pore 450SA | 2.0 |
| Polysorbate 20 | 0.50 |
| Total | 100.00 |

To prepare a compound using Example 1, the water should be heated to 80° C. Add the glycerin, potassium sorbate, allantoin and botanical extracts and mix until all are dissolved. Cool the mixture to 40° C. and add the Sepiplus 400 slowly while mixing to assure that the mixture is free of lumps. Next, slowly add the Poly-Pore polymer to which is supplied with a loading level of 50% salicylic acid. Continue mixing and add the polysorbate. Continue mixing until the compound is uniform. Cool and package. This example would be considered a 1% concentration of salicylic acid.

### EXAMPLE 2

| Ingredients | Weight % |
|---|---|
| Water | 87.90 |
| Glycerin | 5.00 |
| Potassium Sorbate | 0.10 |
| Allantoin | 0.10 |
| Botanical Extracts | 1.00 |
| Sepiplus 400 | 1.20 |
| Poly-Pore 450SA | 4.00 |
| Dry Flo AF 28-1805 | 0.20 |
| Polysorbate 20 | 0.50 |
| Total | 100.00 |

To compound Example 2, as before, first heat the water to 80° C. and add the next four ingredients, stirring to get a smooth, lump free mixture. The mixture is then cooled to 40° C. at which time the Sepiplus is added slowly, while mixing to keep the mixture free of lumps. While continuing to mix, add, in order, the remaining ingredients until a uniform mixture results which can then be packaged as a 2% salicylic acid product.

### EXAMPLE 3

| Ingredients | Weight % |
|---|---|
| Water | 93.19 |
| Glycerin | 2.00 |
| Potassium Sorbate | 0.10 |
| Allantoin | 0.10 |
| Botanical Extracts | 1.10 |
| Sepiplus 400 | 1.00 |
| Poly-Pore 450SA | 2.00 |
| Polysorbate 20 | 0.50 |
| Zincidone | 0.01 |
| Total | 100.00 |

### EXAMPLE 4

| Ingredients | Weight % |
|---|---|
| Water | 91.84 |
| Glycerin | 3.50 |
| Potassium Sorbate | 0.11 |
| Allantoin | 0.10 |
| Extract of Wild Pansy | 0.50 |
| Extract of Licorice | 0.20 |
| Extract of Alfalfa | 0.10 |
| Extract of Orange Peel | 0.15 |
| Sepiplus 400 | 1.00 |
| Poly-Pore 450SA | 2.00 |
| Polysorbate 20 | 0.50 |
| Total | 100.00 |

As with Examples 1 and 2, Examples 3 and 4 are prepared by initially heating the water to 80° C. and adding the glycerine, potassium sorbate, allantoin and the botanical extracts while stirring to achieve a smooth mixture. The mixture is cooled to 40° C. and the remaining ingredients are added, one at a time while stirring to maintain a smooth, lump free mixture which can then be packaged.

### EXAMPLE 5

| Ingredients | Weight % |
|---|---|
| Water | 79.43 |
| Glycerin | 5.00 |
| Ajidew N-50 | 0.20 |
| Potassium Sorbate | 0.10 |
| Allantoin | 0.10 |
| Multifruit BSC | 0.50 |
| Witch Hazel Water | 12.00 |
| Actiphyte of Blue Malva | 0.30 |
| Sepiplus 400 | 1.00 |
| Poly-Pore 450SA | 2.00 |
| Polysorbate 2D | 0.50 |
| Peppermint Oil | 0.25 |
| Citric Acid | 0.25 |
| Total | 100.00 |

### EXAMPLE 6

| Ingredients | Weight % |
|---|---|
| Water | 79.43 |
| Glycerin | 5.00 |
| Potassium Sorbate | 0.10 |
| Witch Hazel Water | 10.00 |
| Ajidew N-50 | 0.30 |
| Allantoin | 0.10 |
| Extract of Licorice | 0.10 |
| Extract of Alfalfa | 0.10 |
| Extract of Wild Pansy | 0.12 |
| Extract of Hops | 0.13 |
| Lactic Acid | 0.10 |
| Bioecolia BP | 0.10 |
| Sepiplus 400 | 1.00 |
| Poly-Pore 450SA | 3.00 |
| Vitamin E Acetate | 0.01 |
| Vitamin A Palmitate | 0.01 |
| Polysorbate 20 | 0.40 |
| Total | 100.00 |

To create the compound of Example 5, the process of Examples 1-4 can be followed through the step of adding the Poly-Pore 450SA and stirring for a uniform mixture. For Example 5, the Polysorbate and peppermint oil are combined in a separate container and, when thoroughly mixed, are added to the compound, while continuing the stirring. Finally, the citric acid is added and the mixing is continued until the mixture is uniform and remains lump-free.

The compounding of Example 6 follows substantially the same procedure. The steps of Examples 1-5 are followed through to the step of adding the Poly-Pore 450SA. As in with the process of Example 5, the Vitamin E Acetate and Vitamin A Palmitate are combined with the Polysorbate in a separate container and that mixture is added to the existing mixture. Stirring is continued until the resulting combination is smooth and ready for packaging with a salicylic acid concentration of 1.5%.

Various concentrations of salicylic acid can be added to the Poly-Pore® polymer so that each of the examples can have salicylic acid concentrations varying from 0.5% to at least 2.0%. Greater concentrations are possible through the use of greater percentages of the Poly-Pore 450SA, but may be contraindicated for a skin lotion. Those skilled in the art may create still other compositions Accordingly, the scope of the invention should be limited only by the scope of the claims appended hereto.

1. A toning lotion comprising:
a. an allyl methacrylate copolymer including adsorbed salicylic acid;
b. a surfactant;
c. a rheological modifier
thereby providing a stable, low viscosity light textured medicating toning lotion.

2. A toning lotion according to claim 1 wherein the concentration by weight of said allyl methacrylate copolymer ranges from 2 to 4 percent.

3. A toning lotion according to claim 1 wherein the concentration by weight of said surfactant is approximately 0.5 percent.

4. A toning lotion according to claim 1 wherein the concentration by weight of said rheological modifier is approximately 1 percent.

5. A toning lotion according to claim 1 further including a humectant whose concentration by weight is between 2 and 5 percent.

6. A toning lotion according to claim 1 wherein the concentration by weight of said surfactant is approximately 0.5 percent.

7. A toning lotion according to claim 1 further including a moisturizer skin protectant whose concentration by weight is approximately 0.1 percent.

8. A toning lotion according to claim 1, further comprising a preservative.

9. A toning lotion according to claim 8 wherein the concentration by weight of said preservative is approximately 0.1 percent.

10. A toning lotion according to claim 1, further comprising a hydrophobically modified polymer to provide a smooth “feel”.

11. A toning lotion according to claim 10 wherein the concentration by weight of said hydrophobically modified polymer is approximately 0.2 percent.

12. A toning lotion according to claim 1, further comprising an antibacterial oil controlling moisturizer.

13. A toning lotion according to claim 1 wherein the concentration by weight of said antibacterial oil controlling moisturizer is approximately 0.01 percent.

14. A toning lotion according to claim 1, further comprising a bacterial growth inhibitor.

15. A toning lotion according to claim 14 wherein the concentration by weight of said bacterial growth inhibitor is approximately 0.1 percent.

16. A toning lotion according to claim 1, further comprising a sunblock.

17. A toning lotion according to claim 16 wherein the concentration by weight of said sunblock is approximately 0.01 percent.

18. A toning lotion according to claim 1, further comprising a retinol precursor.

19. A toning lotion according to claim 18 wherein the concentration by weight of said retinol precursor is approximately 0.01 percent.

20. A toning lotion according to claim 1, further comprising botanical extracts.
